(19) United States
(12) Patent Application Publication
(10) Pub. No.: US 2026/0210922 A1
(43) Pub. Date: Jul. 23, 2026

(54) ELECTRONIC NOSE FOR RAPID DETECTION

(71) Applicant: Ainos, Inc., San Diego, CA (US)

(72) Inventors: Chun-Hsien TSAI, Zhubei City (TW); Chia-Nan LIAO, Zhubei City (TW)

(21) Appl. No.: 19/197,625

(22) Filed: May 2, 2025

(30) Foreign Application Priority Data
Jan. 21, 2025 (TW) 114102576

(51) Int. Cl. G01N 33/00; B01D 53/00; B01D 53/04; B01D 53/86; B01D 53/88; G01N 27/12

(52) U.S. Cl. CPC G01N 33/0026; B01D 53/007; B01D 53/0407; B01D 53/885; G01N 27/12; B01D 53/8696; B01D 2253/102; B01D 2255/802; B01D 2259/804

(57) ABSTRACT

An electronic nose for rapid detection comprises a gas intake unit, a detection unit, a purification unit, an evacuation unit, and a control system. The gas intake unit is used to introduce an external gas. The detection unit includes a chamber, a circulation conduit, and a detection module. The circulation conduit is configured to deliver a circulating gas flow to clean the chamber. The gas intake unit is configured to deliver the external gas to be detected. The purification unit is arranged in the chamber and includes a support, a photocatalyst, and a light-emitting element. The evacuation unit is connected to the circulation conduit to generate negative pressure in the chamber to form the circulating gas flow. The control system is connected to the detection module and receives a detection signal generated by the detection module.

### FIELD OF THE INVENTION

The present invention relates to an electronic nose, particularly an electronic nose capable of rapid detection and suitable for integration into robots.

### BACKGROUND OF THE INVENTION

Robots are widely applied in modern society in various fields including factory automation, home care, environmental exploration, disaster rescue, security patrolling, and gas detection. Typically, robots are equipped with a variety of sensing devices to detect their surroundings and take appropriate measures. Among these sensing devices, an electronic nose is capable of distinguishing and quantifying both simple and complex odors. It uses gas sensors to detect gases in the environment, performs comparisons and analyses, and thereby realizes multiple functions. For example, it can detect harmful gases and issue alerts, monitor air quality, detect dangerous conditions such as fires or gas leaks, be applied in disease and public health, or be used for food analysis.

In some existing electronic nose technologies, such as non-optical gas sensors, continuous environmental monitoring requires repeated calibration of the gas sensors and execution of gas detection processes. This occurs regardless of whether the environmental gas changes, resulting in time-consuming identification procedures that fail to reflect real-time variations in the gas environment. Furthermore, constantly performing these identification processes consumes significant electrical power and shortens the operational lifespan of the electronic nose.

Moreover, in conventional electronic noses, it is typically necessary to clean or purify the internal chamber with clean air beforehand, often by equipping the gas intake with a filtration device. Regarding the choice of filtration device, using commonly applied activated carbon introduces the need for periodic replacement. Alternatively, employing a molecular sieve raises preservation challenges, such as requiring additional waterproofing and vacuum protection. Consequently, gas purification techniques in electronic noses present room for improvement.

### SUMMARY OF THE INVENTION

In at least one example of the present disclosure, an electronic nose equipped with a gas exchange system is provided to analyze external gas. The electronic nose includes a gas intake unit, a detection unit, a purification unit, an evacuation unit and a control system. The gas intake unit is configured to introduce an external gas. The detection unit includes a chamber, a circulation conduit and a detection module. An intake port of the chamber is in fluid communication with the gas intake unit and collectively defining a gas intake channel. The circulation conduit is connected between a discharge port of the chamber and the intake port. The circulation conduit defines a circulation channel. The detection module includes a gas sensor device and one or more environmental sensor devices. The gas sensor device is configured to detect gas within the chamber and generate a detection signal responsive to the gas in the chamber, and the environmental sensor devices are configured to detect one or more environmental parameters of the chamber. The circulation channel is configured to deliver a circulating airflow for cleaning the chamber, and the gas intake channel is configured to deliver the external gas to be detected. The purification unit is disposed within the chamber and positioned such that the circulating airflow passes therethrough. The purification unit includes a support, a photocatalyst disposed on the support, and a light-emitting element configured to activate the photocatalyst on the support. The purification unit is configured to convert the circulating airflow into a purified gas flow. The evacuation unit is connected to the circulation conduit to guide gas from the discharge port of the chamber back to the intake port. The control system is connected to the detection module and configured to receive the detection signal generated by the detection module, and further configured to obtain gas-related information associated with the external gas based on the detection signal generated in response to the external gas entering the chamber from the gas intake channel.

### BRIEF DESCRIPTION OF THE DRAWINGS

Example embodiments of the present disclosure will be described with reference to the accompanying drawings briefly described below.

FIG. 1 is a schematic diagram of a robot according to an embodiment of the present invention.

FIG. 2A is a schematic block diagram illustration of an electronic nose, according to various exemplary embodiments.

FIG. 2B is a schematic block diagram illustration of an electronic nose, according to various exemplary embodiments.

FIG. 2C is a schematic block diagram illustration of an electronic nose, according to various exemplary embodiments.

FIG. 3 is a schematic block diagram illustration of an electronic nose, according to other exemplary embodiments.

FIG. 4 is a schematic flow-chart illustration of a method for operation of an electronic nose, according to various exemplary embodiments.

FIG. 5 shows resistance variation of the detection signal in a monitoring mode, according to various exemplary embodiments.

FIG. 6 shows resistance variation of the detection signal in an identification mode, according to various exemplary embodiments.

### DETAILED DESCRIPTION

It should be understood that the terminology used in the description of various embodiments is for illustration only and is not intended to be limiting. Unless otherwise explicitly stated by context or the number of components is deliberately restricted, the singular terms such as “a” or “the” also include plural forms. Furthermore, the terms “including” and “comprising” indicate the presence of the stated features, components, and/or assemblies without excluding the addition or presence of one or more other features, components, assemblies, or their combinations. Indefinite and definite articles are intended to include both singular and plural meanings unless the context clearly indicates otherwise.

The present invention discloses an electronic nose. In one embodiment, the electronic nose is suitable for mounting on a robot, which may be an autonomous mobile robot, an automated guided vehicle, an articulated robot, a humanoid robot, a collaborative robot, or a hybrid robot. It may also be a mechanical robot or a bionic robot. Nonlimiting examples include patrolling robots, exploration robots, and home care robots. Although examples are provided, the term “robot” is to be interpreted broadly.

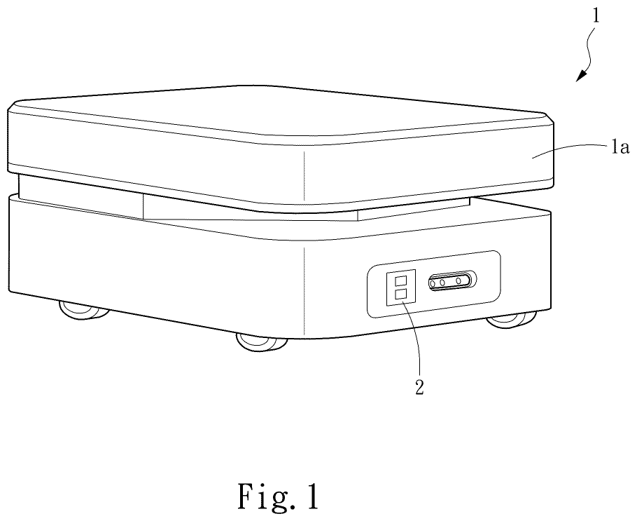

FIG. 1 illustrates a robot according to an embodiment of the present invention. The robot 1 is a wheeled robot equipped with an electronic nose 2. The robot 1 includes a robot body 1a, on which the electronic nose 2 is mounted. At least a portion of the electronic nose 2 is exposed from a casing of the robot 1 so as to be exposed to atmospheric/ambient environment for real-time detection. With the electronic nose 2, the robot 1 can continuously monitor changes in the surrounding gas environment and take necessary actions based on the detection results. In the present disclosure, “external gas” refers to the ambient gas in the space where the robot 1 or the electronic nose 2 is located.

For example, in factory or home environments, there may be excessive amounts of harmful gases such as carbon monoxide, carbon dioxide, ozone, sulfur dioxide, nitrogen dioxide, volatile organic compounds, formaldehyde, and the like. The robot 1 may use the electronic nose 2 to detect whether these harmful gases are present and whether their concentrations exceed a safety standard, thereby generating alerts or activating a ventilation system to enhance gas exchange with the external environment. Alternatively, in unknown or extreme environments such as deep-sea, caves, or space, a mobile robot 1 equipped with the electronic nose 2 can perform real-time analysis of the gas composition in the environment.

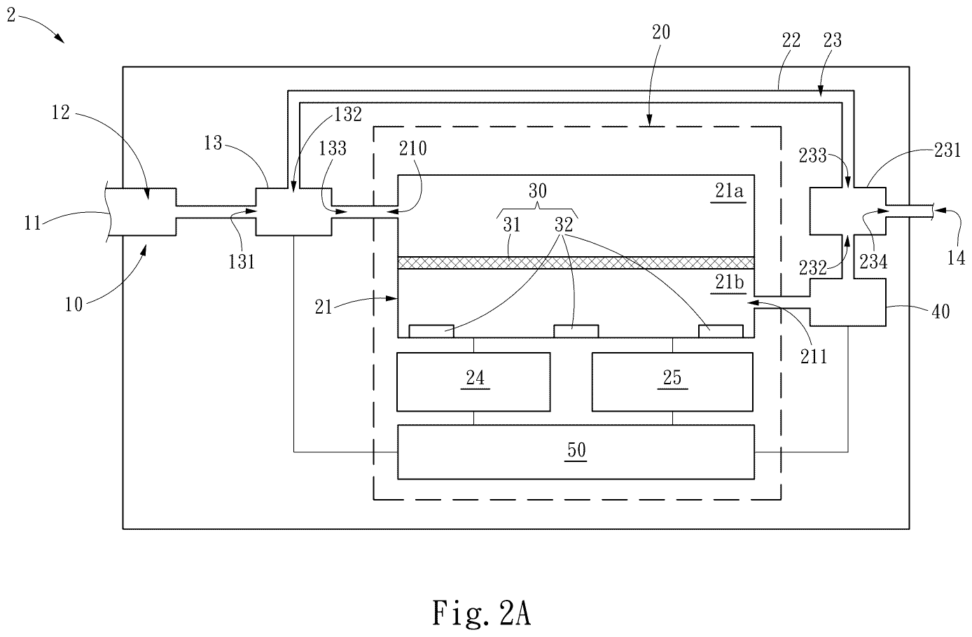

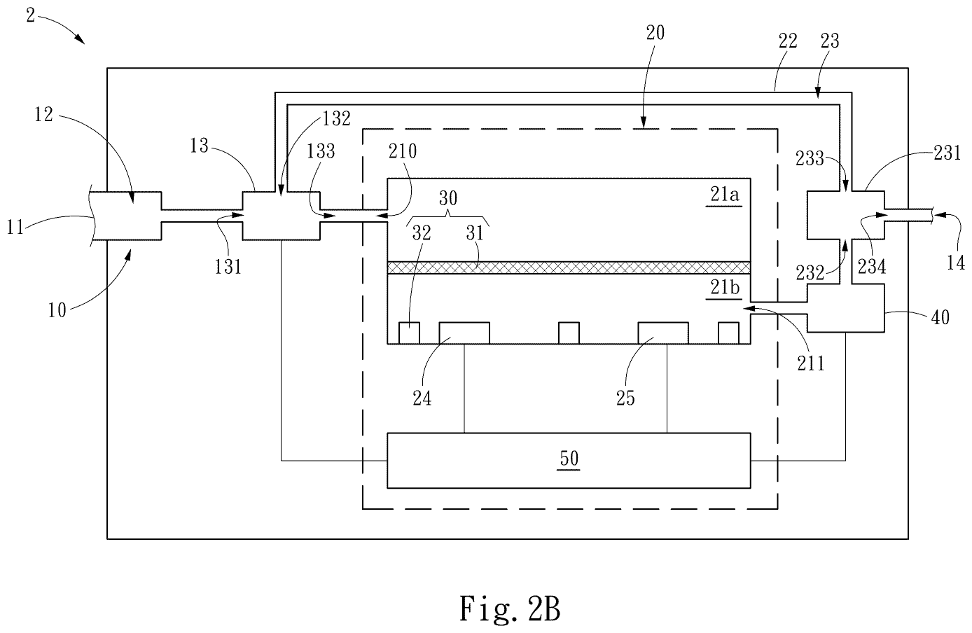

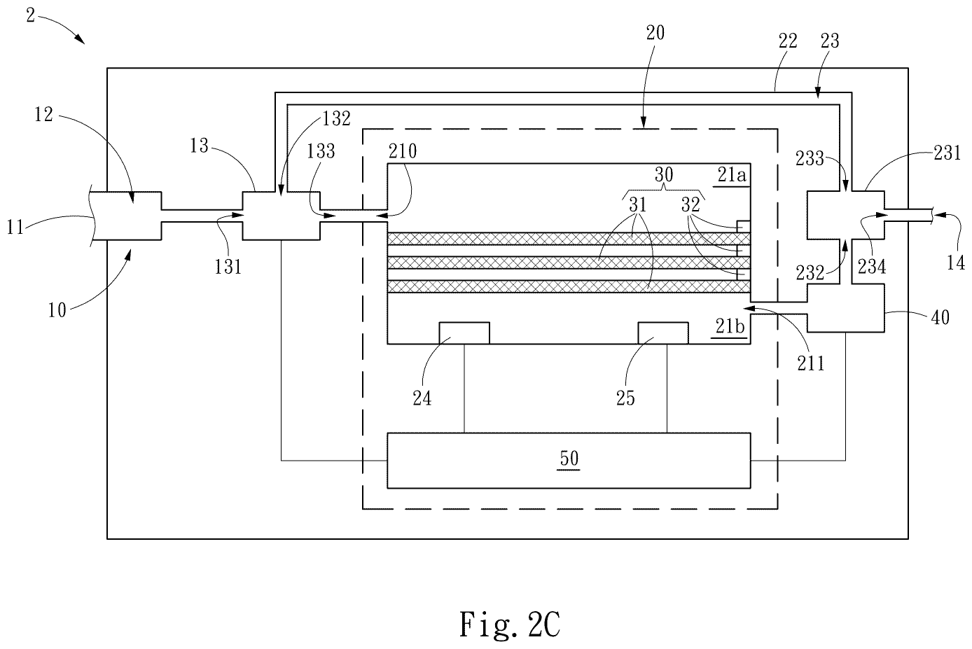

Referring to FIG. 2A, according to an example of the present invention, the electronic nose 2 includes a gas intake unit 10, a detection unit 20, a purification unit 30, an evacuation unit 40, and a control system 50.

The gas intake unit 10 has a gas inlet 11 in communication with the external environment, and the gas intake unit 10 defines a gas intake channel 12. In one example, the gas inlet 11 is disposed on the casing of the robot 1, allowing contact with and entry of external gas into the electronic nose 2. The detection unit 20 includes a chamber 21 and a detection module, where the detection module includes multiple identical or different sensors. The detection module may be located inside the chamber 21. In other examples, the detection module may be located at another position as long as it is capable of detecting the gas within the chamber 21. For example, the detection module may be at least partially exposed to the chamber 21. The chamber 21 includes an intake port 210 and a discharge port 211. An upstream portion of the chamber 21 is fluidly connected to the gas intake unit 10 via the intake port 210 to receive external gas introduced from the gas intake unit 10, while a downstream portion of the chamber 21 is fluidly connected to the evacuation unit 40 via the discharge port 211 to expel gas from the chamber 21. The external gas may be introduced from the gas intake unit 10 into the chamber 21 and subsequently discharged through the discharge port 211 by negative pressure generated by the evacuation unit 40. Additionally, the intake port 210 and the discharge port 211 of the chamber 21 are connected via a circulation conduit 22, which defines a circulation channel 23 therein.

Gas flow through the gas intake channel 12 of the gas intake unit 10 is controlled by a first fluid regulating device 13, while gas flow through the circulation channel 23 of the circulation conduit 22 is controlled by a second fluid regulating device 231. The first fluid regulating device 13 and the second fluid regulating device 231 may regulate both the flow rates of gas through the gas intake channel 12 and the circulation channel 23 and whether gas flows through these channels. In this example, the circulation channel 23 is connected to the gas intake channel 12 and further to the intake port 210. Both the first fluid regulating device 13 and the second fluid regulating device 231 are three-way fluid regulating devices, such as three-way valves. The first fluid regulating device 13 has a first opening 131, a second opening 132, and a third opening 133, which are respectively connected to the gas inlet 11, a downstream end of the circulation conduit 22, and the intake port 210 of the chamber 21. The second fluid regulating device 231 has a first opening 232, a second opening 233, and a third opening 234, which are respectively connected to the discharge port 211 of the chamber 21, an upstream end of the circulation conduit 22, and a gas outlet 14. Accordingly, gas can be selectively allowed to enter from the gas inlet 11, pass through the intake port 210 into the chamber 21 (by opening the first opening 131 and third opening 133 of the first fluid regulating device 13 and closing its second opening 132), and exit from the discharge port 211 of the chamber 21 through the gas outlet 14 (by opening the first opening 232 and third opening 234 of the second fluid regulating device 231 and closing its second opening 233). Alternatively, gas may flow from the discharge port 211 of the chamber 21 through the circulation channel 23 (by opening the first opening 232 and second opening 233 of the second fluid regulating device 231 and closing its third opening 234) and return to the chamber 21 via the intake port 210 (by opening the second opening 132 and third opening 133 of the first fluid regulating device 13 and closing its first opening 131).

The purification unit 30 includes a support 31 and a light-emitting element 32. The support 31 may be a mesh or other gas-permeable components, such as a filter screen. One or more photocatalysts are disposed on the support 31, and the light-emitting element 32 is configured to illuminate the photocatalyst on the support 31. In one example, the photocatalyst may include titanium dioxide (TiO₂), zinc oxide (ZnO), manganese dioxide (MnO₂), iron oxide (Fe₂O₃), or a combination thereof, or it may be a composite photocatalyst material, such as a combination of the aforementioned photocatalyst materials with silver, graphene, or carbon nanotubes. The light-emitting element 32 may emit ultraviolet light, such as deep ultraviolet light (UVC). In one example, the support 31 is disposed within the chamber 21. In one example, the purification unit 30 provides both filtration and purification functions: the support 31 alone filters the gas passing through, while the light-emitting element 32, in conjunction with the photocatalyst, purifies the gas. In some examples, multiple purification units 30 may be present, with the supports 31 spaced apart within the chamber 21. A single light-emitting element 32 may illuminate the photocatalysts on these supports 31, or a plurality of the light-emitting element 32 may be positioned adjacent to each of the supports 31, as shown in FIG. 2C, where the supports 31 are arranged in a multilayer configuration.

In another example, in addition to the photocatalyst, activated carbon may be disposed on the support 31. The activated carbon can also purify the gas passing through, offering rapid adsorption properties. When illuminated, the photocatalyst can decompose substances adsorbed by the activated carbon, preventing it from becoming saturated. This significantly reduces purification time and mitigates the frequent replacement issues associated with activated carbon.

The evacuation unit 40 is configured to generate negative pressure within the chamber 21 for gas extraction and is connected at a position upstream of the second fluid regulating device 231. The evacuation unit 40 may be a vacuum pump.

The chamber 21 includes an upstream region 21a and a downstream region 21b. The upstream region 21a is connected to and proximate the intake port 210, while the downstream region 21b is connected to and proximate the discharge port 211. When the evacuation unit 40 is activated, creating negative pressure within the chamber 21, gas flows from the upstream region 21a to the downstream region 21b. The support 31 is positioned along the gas flow path within the chamber 21, and the light-emitting element 32 may be located inside the chamber 21 or at a position capable of illuminating the photocatalysts on the support 31

The detection module includes a gas sensor device 24 and one or more environmental sensor devices 25. The gas sensor device 24 and the environmental sensor devices 25 may be positioned within the chamber 21 or at least partially exposed to the chamber 21, as shown in FIGS. 2B and 2C. However, this arrangement is not limiting, and the detection module may be placed at other locations capable of contacting and detecting gas within the chamber 21. For example, the detection module may be at least partially exposed to the chamber 21. In one embodiment, the gas sensor device 24 is a device that generates or varies an electrical signal in response to gas, such as a chemical resistance-type or electrochemical gas sensor array or a semiconductor gas detector. The present invention is not limited to these examples, the gas sensor device 24 may also be implemented using other forms or structures, such as optical or electrochemical gas sensors.

The gas sensor device 24 is configured to detect gas or changes in gas within the chamber 21. This may involve detecting the type of gas present, the existence of one or more specific components, the concentration or quantity of a specific component (or whether it reaches a certain value), whether the gas in the chamber 21 conforms to a specific composition, or changes in specific components, composition, or concentration. Specific components may include oxygen, carbon monoxide, hydrogen sulfide, ammonia, chlorine, ozone, sulfur dioxide, nitrogen dioxide, natural gas, liquefied petroleum gas, methane, or propane. Specific compositions may include toxic or combustible gases.

The gas sensor device 24 detects gas in the chamber 21 and generates a detection signal responsive to the gas present. If a chemical resistance-type gas sensor array is used, the detection signal represents a resistance value (e.g., changing from zero to a certain value) or a change in resistance value (e.g., from an initial first value to a subsequent second value). The detection signal may then be used to derive gas-related information related to the external gas. The gas-related information may indicate the presence of one or more specific components in the external gas, the concentration or quantity of the specific component (or whether it reaches a certain value), whether the external gas conforms to a specific composition, or changes in the specific components, composition, or concentration of the external gas.

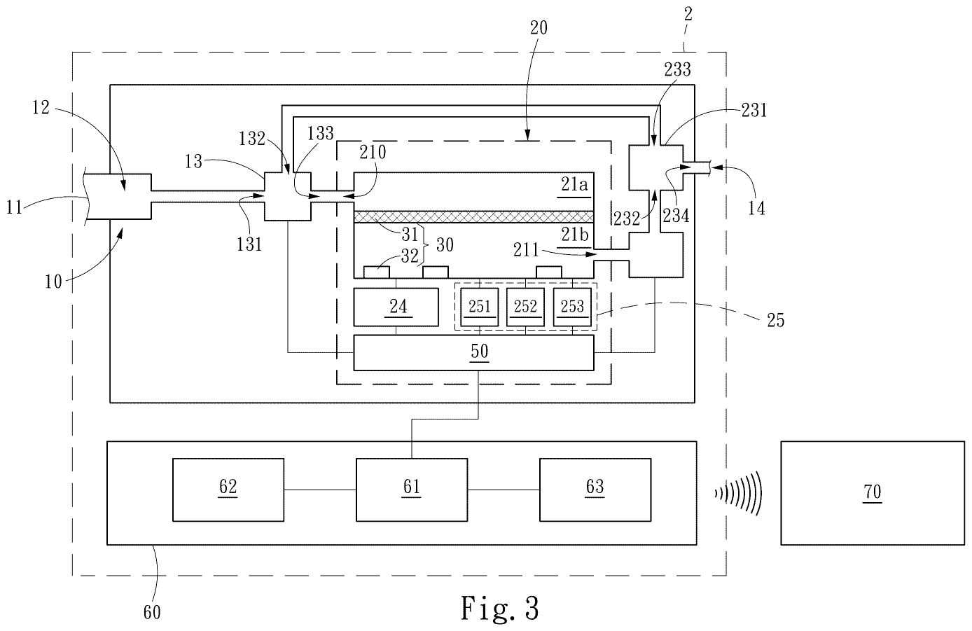

The environmental sensor devices 25 are configured to detect one or more environmental parameters within the chamber 21, which may include temperature, humidity, atmospheric pressure, or any combination thereof. Referring to FIG. 3, depending on the environmental parameters to be detected, the environmental sensor devices 25 may include a temperature sensor 251, a humidity sensor 252, a pressure sensor 253, or any combination thereof. The temperature sensor 251, humidity sensor 252, and pressure sensor device 253 measure the temperature, humidity, and pressure within the chamber 21, respectively.

The control system 50 may control the switching and adjustment of the first fluid regulating device 13 and the second fluid regulating device 231, as well as the activation and deactivation of the evacuation unit 40. Additionally, the control system 50 may control the light-emitting element 32 of the purification unit 30 and is connected to the gas sensor device 24 and/or the environmental sensor devices 25. The control system 50 operates the first fluid regulating device 13 and the second fluid regulating device 231 to selectively enable two gas flow modes: a circulation flow and a single-pass flow (non-recirculating flow).

In the circulation flow mode, gas flows from the discharge port 211 of the chamber 21 through the circulation channel 23 (by opening the first opening 232 and second opening 233 of the second fluid regulating device 231 and closing the third opening 234) and returns to the chamber 21 via the intake port 210 (by opening the second opening 132 and third opening 133 of the first fluid regulating device 13 and closing the first opening 131), forming a circulating airflow.

In the single-pass flow mode, there is no recirculation. Gas enters from the gas inlet 11, passes through the intake port 210 into the chamber 21 (by opening the first opening 131 and third opening 133 of the first fluid regulating device 13 and closing the second opening 132), and exits from the discharge port 211 of the chamber 21 through the gas outlet 14 (by opening the first opening 232 and third opening 234 of the second fluid regulating device 231 and closing the second opening 233).

In the example shown in FIG. 3, the electronic nose 2 is mounted on the robot 1 in a modular manner, and the control system 50 may be further connected to a control unit 60 of the robot 1. The control unit 60 may include a processor 61, a database 62, and a transmission interface 63. The control unit 60 may be used to control the control system 50, receive signals from the control system 50, or serve as a connection pathway between the electronic nose 2 and other external components. In one example, the processor 61 receives and processes the detection signal from the gas sensor device 24 and the environmental parameters from the environmental sensor devices 25. For instance, it may compare the detection signal with data stored in the database 62 and generate an analysis result related to the external gas. In one example, the processor 61 31 may perform artificial intelligence computations, enabling the robot 1 to locally process the detection signal and/or environmental parameters using generative artificial intelligence. In other examples, the control unit 60 may connect to an external device 70, such as a server or an external database, via the transmission interface 63. The transmission interface 63 may support wired or wireless communication protocols, such as WiFi, BLE, Bluetooth, Z-Wave, USB, or Zigbee. It should be understood that in other examples, the control unit 60 may be integrated with the electronic nose 2 as a single module, not limited to the configurations described above.

In the detection process of the electronic nose 2, one of the primary sources of electrical power consumption is the operation of the evacuation unit 40. If the evacuation unit 40 remains active whenever the electronic nose 2 is operational, continuously drawing external gas into the chamber 21, it results in high electrical power consumption.

This shortens the operational duration of the electronic nose 2 and reduces the lifespan of the evacuation unit 40. To address this, the present invention proposes operating the electronic nose 2 in either a monitoring mode or an identification mode. The monitoring mode can be regarded as a phase with lower detection accuracy but reduced electrical power consumption, while the identification mode is a phase with higher detection accuracy and greater electrical power consumption. In one example, the electronic nose 2 operates normally in the monitoring mode by default and remains in this mode continuously until a designated condition is met, at which point the electronic nose 2 switches to the identification mode. In the example, the designated condition may be a change of the external gas requiring further determination or analyzed.

The monitoring mode involves multiple cycles of a standby time and short-duration gas intake. The monitoring mode continues until the detection signal during the short-duration gas intake meets a specific condition, triggering a switch from the monitoring mode to the identification mode. The identification mode involves a single cycle of long-duration gas intake followed by a detection. It should be understood that the term “short-duration” is relative to “long-duration,” and within these modes, the time periods may be the same or different. For example, the standby time is greater than the duration of the short-duration gas intake. By adjusting the ratio of the short-duration to the long-duration periods (or periods of the short-duration and the long-duration), as well as the ratio of the standby time to the duration of the short-duration gas intake (or periods of the standby time and the short-duration gas intake), the operational time of the evacuation unit 40 during the overall detection process of the electronic nose 2 can be significantly reduced. This not only saves electrical power consumption but also extends the lifespan of the evacuation unit 40.

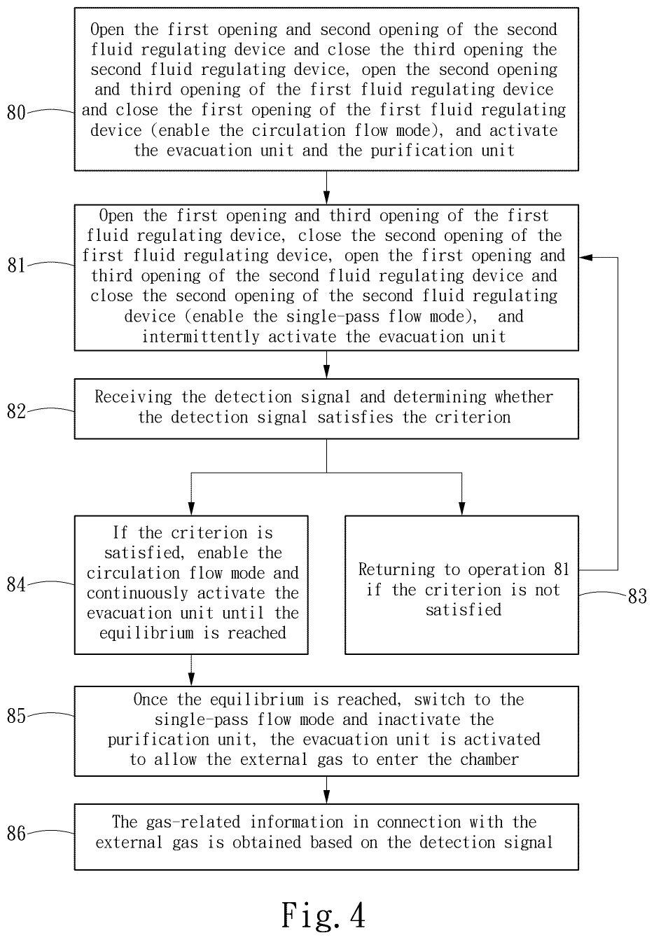

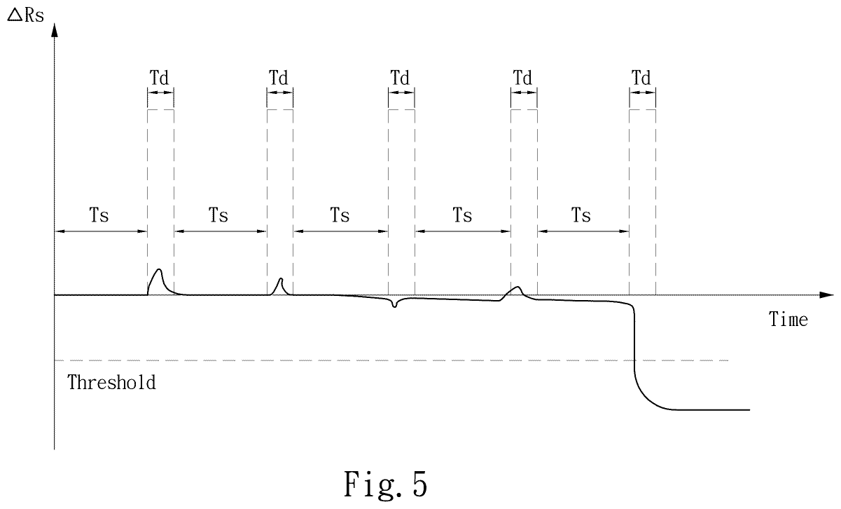

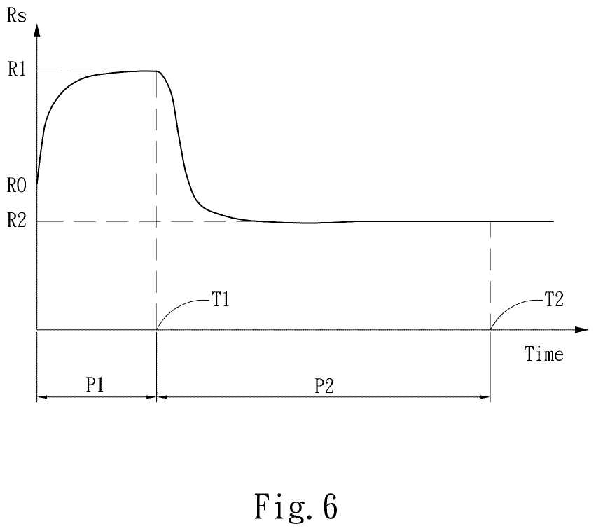

In the following example, the gas sensor device 24 is a chemical resistance-type gas sensor, and the detection signal is a resistance value. Referring to FIG. 4, which illustrates the operational flow of the electronic nose 2, along with FIGS. 5 and 6, the electronic nose 2 normally operates in the monitoring mode by default and switches to the identification mode only when the specific condition is met. In this example, before entering the monitoring mode, the electronic nose 2 operates in the circulation flow mode with the purification unit 30 activated (i.e., the light-emitting element 32 is turned on). The circulating airflow of purified gas continuously and repeatedly flows through the chamber 21 via the circulation channel 23 until the resistance value obtained from the gas sensor device 24 becomes stabilized (Operation 80), at which point the monitoring mode begins. The purpose of Operation 80 is to clean the chamber 21 and/or bring the chamber 21 to an equilibrium before detection. Since the gas passes through the support 31 before flowing over the chamber 21, the gas flowing near the gas sensor device 24 is purified gas. In Operation 80, the evacuation unit 40 and the purification unit 30 are activated continuously, without breaks.

Subsequently, the electronic nose 2 enters the monitoring mode, where gas flow adopts the single-pass flow mode. The electronic nose 2 cycles through periods of standby time and short-duration gas intake. During the standby time, the evacuation unit 40 and the light-emitting element 32 are not activated, so no gas is drawn into the chamber 21, and no detection occurs. During the short-duration gas intake, the evacuation unit 40 is activated (while the light-emitting element 32 remains off). At this time, with the circulation channel 23 closed, external gas to be detected is drawn into the chamber 21 from the gas inlet 11 via the intake port 210, passes through the support 31 and the gas sensor device 24 (Operation 81), and exits through the discharge port 211 to the gas outlet 14. In Operation 81, the evacuation unit 40 operates intermittently, with alternating on and off cycles, where the duration of each cycle may be consistent or variable, and the purification unit 30 is inactivated. Since the light-emitting element 32 is off, the gas detected by the gas sensor device 24 is unpurified gas. When gas flows past the gas sensor device 24, it is detected (Operation 82).

FIG. 5 illustrates the change in the resistance value (ΔRs) of the gas sensor device 24 over time during the monitoring mode, where ΔRs=R_{S(t)}−R_{S(t-1)}. Here, R_{S(t)} represents the resistance value at time t, and R_{S(t-1)} represents the resistance value at the previous time point t−1, with the time interval between t and t−1 chosen according to requirements (for instance, t could represent the 5th second while t−1 might represent the 3rd second). The monitoring mode includes multiple standby time periods Ts (the standby time) and multiple detection time periods Td (the short-duration gas intake), with the detection time period Td following the standby time period Ts. During the standby time period Ts, the evacuation unit 40 does not draw gas, and the evacuation unit 40 only operates during the shorter detection time period Td. In other words, since no external gas is introduced into the chamber 21, the resistance value Rs undergoes only negligible variation, as illustrated in the five Ts segments in FIG. 5. During the detection time period Td, external gas is introduced into the chamber 21, causing the resistance value to change, as shown in the five Td segments in FIG. 5.

If the change in the resistance value (ΔRs) induced by the external gas is small or below a threshold, the electronic nose 2 continues operating in the monitoring mode (Operation 83), as shown in the first four Td segments in FIG. 5. However, if the change in the resistance value ΔRs induced by the external gas is significant or exceeds the threshold, the electronic nose 2 switches to the identification mode, as shown in the fifth Td segment in FIG. 5 (Operation 84). In one example, the detection time period Td is shorter than the standby time period Ts. In one example, the ratio of the detection time period Td to the standby time period Ts is between 0 and 1, such as less than ⅕, 1/10, or 1/15. In Operation 84, the evacuation unit 40 and the purification unit 30 are activated continuously, without breaks.

FIG. 6 illustrates the variation of the resistance value (Rs) over time in the identification mode. The identification mode includes two phases: a pre-detection phase P1 and a detection phase P2. In the pre-detection phase P1, the control system 50 configures gas flow in the circulation flow mode, and the purification unit 30 is activated (i.e., the light-emitting element 32 is turned on), allowing purified external gas (purified gas) to enter the downstream region 21b of the chamber 21 (Operation 84). This purified gas is not the gas to be detected but can be regarded as a background gas, a reference gas, or a cleaning gas used to bring the chamber 21 to an equilibrium prior to detection. In some aspects, the pre-detection phase P1 may also be considered a pre-cleaning stage.

When the support 31 is equipped with both the photocatalyst and activated carbon, compared to using only the photocatalyst, the time required for the pre-detection phase P1 and Operation 80 can be further reduced.

The control system 50 receives the detection signal from the gas sensor device 24 and the environmental parameters from the environmental sensor devices 25, observing and determining whether an equilibrium has been reached based on the values of the detection signal and the environmental parameters (Operation 84). The equilibrium may be defined as the condition in which the detection signal and one or more environmental parameters in the chamber 21 have reached a steady value or slightly varied within a range. The equilibrium may be a state that the balanced detection signal and the balanced environmental parameters are consistently sustained under continuous gas flow conditions. The environmental parameters may include temperature, humidity, and/or pressure. It should be understood that the equilibrium state can encompass a single environmental parameter (e.g., temperature alone) or multiple parameters reaching equilibrium, though the more parameters that achieve equilibrium, the more effectively detection can proceed.

“Reaching the equilibrium” means that the detection signal and the environmental parameters in the chamber 21 are substantially constant. In one example, substantially constant may mean varying within a range of a certain value over time, such as within ±10%, ±5%, or ±1%. The control system 50 may determine that the equilibrium is reached if the substantially constant state of the resistance value and the environmental parameters (temperature, humidity, pressure, or any combination thereof) persists for a predetermined threshold duration. In other words, the equilibrium state is a range within the pre-detection phase P1 during which the detection signal and the environmental parameters continuously remain substantially constant. In one embodiment, the evacuation unit 40 is controlled so that the flow rate of the filtered gas entering the chamber 21 remains substantially constant over time, thereby ensuring a stable gas flow within the chamber 21 and facilitating rapid attainment of the equilibrium.

As shown in FIG. 6, during the pre-detection phase P1, the resistance value of the detection signal generated by the gas sensor device 24 gradually increases from an initial resistance R0 and stabilizes at a first resistance value R1 at time T1. At this point, the equilibrium is achieved. The time from the start until T1 is defined as a first time interval.

Once the equilibrium is reached, the electronic nose 2 proceeds to the detection phase P2 (Operation 85). In this example, upon transitioning from the pre-detection phase P1 to the detection phase P2, the control system 50 maintains the operation of the evacuation unit 40 and controls the first fluid regulating device 13 and the second fluid regulating device 231 to enable gas flow in the single-pass flow mode, while deactivating the purification unit 30 (i.e., turning off the light-emitting element 32). As a result, the gas entering the chamber 21 and flowing through the downstream region 21b is unpurified external gas to be detected. That is, the external gas to be detected is unpurified to ensure accurate analysis by the gas sensor device 24. In this example, the evacuation unit 40 operates continuously. During the pre-detection phase P1, the first opening 232 and second opening 233 of the second fluid regulating device 231 are open, and the third opening 234 is closed, while the second opening 132 and third opening 133 of the first fluid regulating device 13 are open, and the first opening 131 is closed. During the detection phase P2, the first opening 131 and third opening 133 of the first fluid regulating device 13 are open, and its second opening 132 is closed, while the first opening 232 and third opening 234 of the second fluid regulating device 231 are open, and its second opening 233 is closed.

As the gas flowing past the gas sensor device 24 changes, as shown in FIG. 6, the resistance value of the detection signal generated by the gas sensor device 24 shifts from the first resistance value R1 to a second resistance value R2, stabilizing at T2 and reflecting one or more properties of the unpurified external gas. The period from the end of T1 (or the start of T2) to the end of T2 is defined as a second time interval. The control system 50 receives the detection signal from the gas sensor device 24 and the environmental parameters from the environmental sensor devices 25 (Operation 86) and derives the gas-related information based on the detection signal. In one example, the above detection is performed at room temperature without heating the gas in the chamber 21. However, the present invention is not limited to this, and in some examples, detection may occur with the gas in the chamber 21 heated, e.g., to temperatures above 50° C. or between 50° C. and 450° C. In the example, the gas-related information is irrelevant to the detection signal of the circulating airflow.

The gas entering chamber 21 during the first time interval is a purified gas to clean the chamber, while the gas entering during the second time interval is the gas to be detected. The evacuation unit 40 operates continuously, without deactivation, throughout both the first and second time intervals. As a result, gas flows uninterrupted into the chamber 21 across both time periods (the first and second time intervals). The second time interval follows immediately after the first time interval without any interruption.

In one embodiment, the equilibrium is in a dynamic mode, which involves two aspects. First, the gas in the chamber 21 is in motion, meaning the gas flows rather than is static. Second, during the pre-detection phase P1, the detection signal and the environmental parameters remain substantially constant for the predetermined threshold period under this flowing gas condition. In one embodiment, when transitioning from the pre-detection phase P1 to the detection phase P2, the evacuation unit 40 remains operational, and gas is continuously introduced into the chamber 21 from either the circulation channel 23 or the external environment without interruption.

In one example, the purified circulating gas within the chamber 21 generates a flowing first gas stream, while the unpurified external gas generates a flowing second gas stream, with the first gas stream during the pre-detection phase P1 and the second gas stream during the detection phase P2 maintaining a substantially identical flow rate. As the evacuation unit 40 is not deactivated during the transition, gas flow in the chamber 21 remains continuous, with only the gas changing. Consequently, the variation in environmental parameters within the chamber 21 is minimized, meaning the equilibrium is less disturbed or disrupted, and the environment within the chamber 21 does not need to be re-established, thereby enhancing measurement accuracy and reducing time. Under these conditions, the equilibrium state can be interpreted as a dynamic equilibrium.

The present invention recognizes that when introducing external gas for detection, it is essential to maintain a stable detection environment, that is, to achieve equilibrium, in order to obtain accurate detection results. Therefore, before introducing unpurified gas to be detected, the environment within the chamber 21 is first maintained in this dynamic equilibrium, where various environmental parameters remain substantially constant despite continuous gas flow. Subsequently, without interrupting the gas flow, the electronic nose 2 switches to introducing the unpurified gas to be detected, maintaining a substantially identical flow rate (i.e., the gas pressure within the chamber 21 remains substantially the same across both phases).

However, in certain aspects of the present invention, the equilibrium state is not necessarily a dynamic equilibrium and may also be a static equilibrium. The difference in this operation lies in deactivating the evacuation unit 40 after the pre-detection phase P1 ends, allowing the detection signal and environmental parameter values to reach the equilibrium state in the absence of gas flow within the chamber 21 before entering the detection phase P2. According to one aspect of the present invention, the electronic nose 2 can be selectively operated in either the dynamic equilibrium mode or the static equilibrium mode.

The electronic nose 2 of the present invention is designed to operate in the monitoring mode by default, entering the identification mode only when specific conditions are met. In the monitoring mode, the operational time of the evacuation unit 40 significantly exceeds its downtime, substantially reducing electrical power consumption and extending the lifespan of the evacuation unit 40.

Furthermore, the electronic gas cleaning technology employed in the present invention eliminates the need for replacement and preservation, and it can be paired with different modes (monitoring mode, identification mode) or phases (pre-detection phase P1, detection phase P2) to perform automated gas detection more efficiently.

What is claimed is:

1. An electronic nose with a gas exchange system, comprising:
a gas intake unit, configured to introduce an external gas;
a detection unit, including a chamber, a circulation conduit, and a detection module, an intake port of the chamber being in fluid communication with the gas intake unit and collectively defining a gas intake channel, the circulation conduit being connected between a discharge port of the chamber and the intake port, the circulation conduit defining a circulation channel, the detection module including a gas sensor device and one or more environmental sensor devices, the gas sensor device being configured to detect gas within the chamber and generate a detection signal responsive to the gas in the chamber, and the environmental sensor devices being configured to detect one or more environmental parameters of the chamber, wherein the circulation channel is configured to deliver a circulating airflow for cleaning the chamber, and the gas intake channel is configured to deliver the external gas to be detected;
a purification unit disposed within the chamber and positioned such that the circulating airflow passes therethrough, the purification unit including a support, a photocatalyst disposed on the support, and a light-emitting element configured to activate the photocatalyst on the support, wherein the purification unit is configured to convert the circulating airflow into a purified gas flow;
an evacuation unit connected to the circulation conduit to guide gas from the discharge port of the chamber back to the intake port; and
a control system connected to the detection module and configured to receive the detection signal generated by the detection module, the control system being further configured to obtain gas-related information associated with the external gas based on the detection signal generated in response to the external gas entering the chamber from the gas intake channel.

2. The electronic nose according to claim 1, wherein the electronic nose is configured to perform the following steps:
Step 1-1: following each standby time period, allowing the external gas to enter the chamber from the gas intake channel for a detection time period, wherein the detection time period is shorter than the standby time period;
Step 1-2: repeating Step 1-1 until the detection signal meets a specific condition;
Step 1-3: when the specific condition is met, close the gas intake channel and open the circulation channel, allowing the gas within the chamber to be discharged from the discharge port, pass through the circulation channel and the intake port, and re-enter the chamber to form the circulating airflow, continuing until the detection signal and the environmental parameters reach an equilibrium; and
Step 1-4: closing the circulation channel and opening the gas intake channel, thereby allowing the external gas to enter the chamber from the gas intake channel, and obtaining the gas-related information based on the detection signal generated from the external gas entering the chamber.

3. The electronic nose according to claim 2, wherein the gas-related information is irrelevant to the detection signal of the circulating airflow.

4. The electronic nose according to claim 2, wherein the light-emitting element is activated during Step 1-1 and Step 1-3, and deactivated during Step 1-4.

5. The electronic nose according to claim 2, wherein the specific condition is that a change in the detection signal generated by the gas sensor device during the detection time period reaches a threshold.

6. The electronic nose according to claim 2, wherein the evacuation unit is deactivated during the standby time period and activated during the detection time period.

7. The electronic nose according to claim 2, wherein under the equilibrium, the detection signal and the environmental parameters continuously remain substantially constant within the period.

8. The electronic nose according to claim 1, wherein the electronic nose is configured to perform the following steps:
Step 2-1: closing the gas intake channel and opening the circulation channel, allowing the gas in the chamber to be discharged from the discharge port, pass through the circulation channel and the intake port, and re-enter the chamber to form the circulating airflow, continuing until the detection signal and the environmental parameters reach an equilibrium; and
Step 2-2: closing the circulation channel and opening the gas intake channel, allowing the external gas to enter the chamber from the gas intake channel, whereby gas-related information associated with the external gas is obtained based on the detection signal generated from the external gas entering the chamber via the gas intake channel.

9. The electronic nose according to claim 8, wherein the gas-related information is irrelevant to the detection signal of the circulating airflow.

10. The electronic nose according to claim 8, wherein the light-emitting element is activated during Step 2-1, and deactivated during Step 2-2.

11. The electronic nose according to claim 8, wherein the equilibrium is defined as a condition in which, over a time interval, the detection signal and the environmental parameters each continuously remain substantially constant.

12. The electronic nose according to claim 8, wherein the Step 2-1 is performed continuously during a first time interval, the Step 2-2 is performed continuously during a second time interval, and the second time interval immediately follows the first time interval without interruption

13. The electronic nose according to claim 12, wherein the circulating airflow flows in the chamber throughout the first time interval is a purified gas to clean the chamber, and the gas entered the chamber throughout the second time interval is a gas to be detected.

14. The electronic nose according to claim 1, wherein the gas-related information is irrelevant to the detection signal of the circulating airflow.

15. The electronic nose according to claim 1, wherein the environmental parameters include the chamber's temperature, humidity, atmospheric pressure, or any combination thereof.

16. The electronic nose according to claim 1, wherein the gas sensor device is a chemical resistance-type gas sensor and the detection signal is a resistance value.

17. The electronic nose according to claim 1, wherein the environmental sensor devices include a temperature sensor, a humidity sensor, a pressure sensor, or any combination thereof.

18. The electronic nose according to claim 1, wherein the processing unit is connected to a control unit, the control unit comprising a processor and a database, the processor being configured to perform artificial intelligence computations based on the database and the detection signal to generate an analysis result.

19. The electronic nose according to claim 1, wherein the purification unit further includes an activated carbon disposed on the support.

20. The electronic nose according to claim 1, wherein the external gas enters the chamber through the gas intake channel is without filtering.
